(19) United States
(12) Patent Application Publication
(10) Pub. No.: US 2006/0211649 A1
(43) Pub. Date: Sep. 21, 2006

(54) Use of cytisine for enhancing physical performance

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave, Detroit, MI, 48226, US

(21) Appl. No.: 11/385,075

(22) Filed: Mar. 21, 2006

Related U.S. Application Data
(60) Provisional application No. 60/663,609, filed on Mar. 21, 2005.
(60) Provisional application No. 60/712,345, filed on Aug. 31, 2005.

(51) Int. Cl. A61K 31/7072; A61K 31/513

(52) U.S. Cl. 514/50; 514/269

(57) ABSTRACT

A method of decreasing fat and adipose tissue, and increasing endurance and alertness in humans comprises administering cytisine in a daily dosage between about 0.1 and 8 mg. Cytisine may be administered once per day or in multiple doses, either alone or in combination with other compounds and mixtures.

### RELATED APPLICATION

This application claims priority benefit of U.S. provisional patent applications No. 60/663,609 filed on Mar. 21, 2005 and No. 60/712,345 filed on Aug. 31, 2005.

### FIELD OF THE INVENTION

This invention relates to use of cytisine as a supplement for enhancing physical performance.

### BACKGROUND OF THE INVENTION

Obesity is a growing concern, particularly in the United States. Excessive body weight can lead to a variety of well known health problems, especially hypertension, heart disease, atherosclerosis, diabetes and strokes. Weight loss, specifically fat loss, is critical in maintaining a healthy body weight. Unfortunately, the sedate modern lifestyle leaves people consuming more calories than they need.

In addition to diet control and exercise, there are several strategies for decreasing body weight through the use of pharmaceuticals and dietary supplements. Sympathomimetics, (stimulators of the sympathetic nerves) like ephedrine and amphetamines have been used extensively for this purpose. Although such compounds have been found effective, they are associated with side effects including increased heart rate and blood pressure.

Other strategies to decrease body weight include the use of compounds such as nicotine. Nicotine is known to stimulate acetylcholine receptors, and nicotine has been shown to have positive effects on weight loss. Unfortunately, the negative side effects of nicotine are well known and include addiction as well as carcinogenicity.

Cytisine has been described for certain applications. For example, U.S. Pat. No. 4,971,079 describes the use of cytisine for the treatment of nicotine addiction. Similarly, U.S. Pat. No. 6,235,734 also discloses the use of cytisine for treating addictive disorders and neurological or mental disorders related to a decrease in cholinergic function. U.S. Pat. No. 6,194,397 discloses the method of utilizing Cytisine derivatives as Analeptic agents for liver protection against hepatotropic agents. It would be desirable to provide a new method for reducing weight, especially fat and adipose tissue, increasing endurance and increasing alertness.

### SUMMARY OF THE INVENTION

In accordance with a first aspect, a method of decreasing fat and adipose tissue and increasing endurance and alertness in humans comprises administering cytisine in a daily dosage between about 0.1 and 8 mg.

From the foregoing disclosure and the following more detailed description of various preferred embodiments it will be apparent to those skilled in the art that the present invention provides a significant advance in the methods for reducing fat and adipose tissue and increasing endurance. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the methods of use of cytisine disclosed here. The following detailed discussion of various alternative and preferred features and embodiments will illustrate the general principles of the invention with reference to improved methods of enhancing physical performance through the use of orally available dietary supplements. Other embodiments suitable for other applications will be apparent to those skilled in the art given the benefit of this disclosure.

The present invention provides a method of administering to mammals, especially humans, cytisine or one of its derivatives, salts, extracts, tinctures or decoctations thereof, to maintaining or increasing endurance and decreasing fat mass.

As used herein, a derivative of a compound refers to a species having a chemical structure that is similar to the compound, yet containing a chemical group not present in the compound and/or deficient of a chemical group that is present in the compound. The substance to which the derivative is compared is known as the “parent” substance. Here, for example, the parent compound is cytisine, or 1,5-Methano-8H-pyrido[1,2-a][1,5]diazocin-8-one. A derivative may be made by modification of the parent compound or by synthesis from other starting materials that are not similar to the parent.

Cytisine C₁₁H₁₄N₂O, is a naturally occurring tricyclic compound which can be derived from several sources including, for example, the herb genera Sophora and Thermopsis. The chemical structure of cytisine is reproduced below.

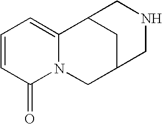

The method of using cytisine to decrease body weight, especially reducing fat and adipose tissue, as well as increasing endurance and alertness is a significant improvement over the use of sympathomimetics and/or nicotine in that it can achieve the positive effects without the addictive side effects.

Administration of cytisine to an individual provides numerous benefits, not just as an anti-obesity agent, but also as a thermogenic, a fatigue reliever and neural stimulant. Cytisine can be administered one of the several ways: orally, transdermally, intranasally, by injection, sublingually, and transrectally as a suppository. Oral administration can comprise, for example, a liquid suspension, gel capsule or tablets. Tablets can be prepared by combining cytisine with conventionally used binders and excipients, e.g. cellulose. Preferably a dosage would be about 0.1-8 mg per day, most preferably about 0.5-2 mg per day. Unit dosage can be about 0.1-4 mg, most preferably 0.2-1 mg of the compound. Oral dosage forms could be administered in smaller divided doses multiple times per day (such as, two or three times per day) to help maintain more constant levels of cytisine in the body.

Other compounds or mixtures may be added in combination with the cytisine. For example caffeine may be mixed in with cytisine or caffeine containing compounds such as tea, most preferably green tea. Green tea is particularly desirable as in addition to caffeine it also has relatively high levels of catechin polyphenols, particularly epigallocatechin gallate (EGCG). EGCG is a powerful anti-oxidant.

### EXAMPLE 1

Capsules. 100 mg of Cytisine is mixed with microcrystalline cellulose, and placed into 1000 hard-gelatin capsules. Each capsule contains 0.1 mg of Cytisine.

### EXAMPLE 2

Capsules. 2 grams Cytisine was mixed with 5 kg of green tea, and placed into 10,000 hard-gelatin capsules. Each capsule contains 0.2 mg of Cytisine and 500 mg of green tea.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of decreasing fat and adipose tissue in humans comprising administering cytisine in a daily dosage between about 0.1 mg and 8 mg.

2. The method of claim 1 wherein cytisine is administered in one of the following ways: orally, transdermally, intranasally, by injection, sublingually, and transrectally.

3. The method of claim 1 wherein cytisine is administered in a daily dosage between about 0.5 mg and 2 mg.

4. The method of claim 1 wherein cytisine is administered in a daily dosage between about 0.1 mg and 4 mg.

5. The method of claim 1 wherein cytisine is administered in a daily dosage between about 0.2 mg and 1 mg.

6. The method of claim 1 wherein cytisine is administered in a dosage between about 0.1 mg and 0.5 mg three times per day.

7. The method of claim 1 wherein cytisine is administered in a dosage between about 0.05 mg and 0.25 mg three times per day.

8. The method of claim 1 wherein the cytisine is administered in the form of one of a gel capsule and a liquid suspension.

9. The method of claim 1 wherein the cytisine is administered in a tablet which also contains cellulose.

10. The method claim 1 wherein the cytisine is administered in combination with caffeine.

11. The method claim 1 wherein the cytisine is administered in combination with green tea.
